(19) United States
(12) Patent Application Publication
(10) Pub. No.: US 2025/0332371 A1
(43) Pub. Date: Oct. 30, 2025

(54) ANESTHESIA SYSTEM

(71) Applicant: Drägerwerk AG & Co. KGaA, Lübeck (DE)

(72) Inventors: Ralf HEESCH, Lübeck (DE); Robert SCHMID, Lübeck (DE); Martin LINNARTZ, Lübeck (DE)

(21) Appl. No.: 19/191,601

(22) Filed: Apr. 28, 2025

(30) Foreign Application Priority Data
Apr. 30, 2024 (DE) 10 2024 112 092.4

(51) Int. Cl. A61M 16/08; A61M 16/00; A61M 16/10; G16H 10/60; G16H 20/10; G16H 40/63

(52) U.S. Cl. CPC A61M 16/0891; A61M 16/0003; A61M 16/0051; A61M 16/009; A61M 16/024; A61M 16/1005; A61M 16/104; G16H 10/60; G16H 20/10; G16H 40/63; A61M 2016/0027; A61M 2016/0033; A61M 2016/1025; A61M 2202/0007; A61M 2202/0208; A61M 2202/0241; A61M 2205/18; A61M 2205/3303; A61M 2205/3327; A61M 2205/3334; A61M 2205/50; A61M 2230/005; A61M 2230/40

(57) ABSTRACT

A device (11) on a circuit system (8) for an anesthesia system (100), is configured to increase an amount of oxygen (O₂) in the circuit system (8) in a special operating situation. The anesthesia system includes a control unit (20) that determines an individual ventilation situation and determines, on the basis of measured values provided by a sensor system (30), whether the special operating situation (74) is present.

### CROSS REFERENCE TO RELATED APPLICATIONS

This application claims the benefit of priority under 35 U.S.C. § 119 of German Application 10 2024 112 092.4, filed Apr. 30, 2024, the entire contents of which are incorporated herein by reference.

### TECHNICAL FIELD

The present invention relates to an anesthesia system. Anesthesia systems are used to safely administer inhalation anesthesia. Modern anesthesia systems have a closed or semi-closed breathing system, often also referred to as a circuit system (breathing circuit system), in which most of the breathing gas does not leave the device. The exhaled carbon dioxide is absorbed by breathing lime and fresh gas is added to the exhaled gas when it is returned to the circuit. This procedure has the advantage that the substances used for anesthesia (general anesthetics) can be used efficiently.

### BACKGROUND

Different variants of anesthesia devices with a radial blower (blower, radial compressor, blower) are described in U.S. Pat. No. 5,875,783 A. U.S. Pat. No. 5,875,783 A shows a circuit system that can be configured with a radial blower. For the purpose of understanding the function and advantages of the circuit system according to the prior art, as described in DE19714644 C2, the functions of the circuit system are described in this application with reference to the description of the figures in FIG. 6 of DE19714644 C2. During inhalation, a radial blower draws in an anesthetic gas in the form of a mixture of oxygen, air, nitrous oxide and vaporized anesthetic from a so-called fresh gas line and also buffered breathing gas from a manual resuscitation bag as inhalation gas. If the pressure level in the patient's lungs is lower than the pressure level at the radial blower, this inhaled gas passes through a carbon dioxide absorber and then through an inspiratory non-return valve via breathing tubes, a patient connecting element (patient Y-piece) and an airway access (breathing mask, endotracheal tube, tracheostoma) to and into the patient. As soon as the pressure conditions are reversed, i.e. as soon as the pressure level in the patient's lungs is above the pressure level at the radial blower, the gas flows from the patient through an expiratory non-return valve back into the manual ventilation bag.

With regard to the mostly used volatile anesthetic gases, it should also be noted here that saving anesthetic gases when performing so-called low-flow anesthesia with low fresh gas flow rates in closed or partially open anesthesia systems brings significant cost savings on the one hand, but also reduces the release of anesthetic gases into the environment.

The reduction of quantities of anesthetic gases released into the environment is also very welcome for reasons of climate protection, as volatile anesthetic gases such as desflurane, isoflurane, enflurane, sevoflurane and halothane can have a similar effect to carbon dioxide or methane as climate-damaging gases. However, when performing anesthesia with low fresh gas flow rates, it must be ensured at all times that the patient is supplied with a sufficient amount of oxygen; in particular, it must be ensured that the oxygen concentration does not fall significantly below the proportion of oxygen in natural ambient air of approx. 21% in all operating states of the anesthesia device or anesthesia system.

### SUMMARY

Based on the state of the art, it is an object of the invention to further develop an anesthesia system in such a way that it can provide a sufficient supply of oxygen—i.e. an optimized oxygen supply-even when using small amounts of fresh gas.

The object is attained by features according to the invention.

The object is attained by an anesthesia system according to the invention.

The invention is explained in more detail in the following description, with partial reference to the figures.

The embodiments disclosed herein create possibilities and variants of an anesthesia system.

Further features and details of the invention and advantageous embodiments are disclosed in the following description, drawings and claims.

The references used here indicate the further development of the subject-matter of the invention and are not to be understood as a waiver of the attainment of independent, subject-matter protection for the feature combinations.

Furthermore, with regard to an interpretation of the claims and the description, if a feature is specified in more detail in a subsequent claim, it must be assumed that such a limitation is not present in the respective preceding claims or in a more general embodiment of the device in question.

Any reference in the description to aspects of subordinate claims is therefore to be read expressly as a description of optional features, even without specific reference.

An anesthesia system according to the invention for performing anesthesia on a living being with an optimized oxygen supply has at least the following components:
a control unit,
a breathing gas supply system comprising a mixing unit, a circuit system, an absorber unit for removing carbon dioxide (CO₂) from the circuit system and a gas conveying unit for supplying quantities of a gas mixture for performing anesthesia on the living being,
a sensor system with
a. at least one pressure sensor P1,
b. at least one gas sensor G1,
c. at least one flow sensor V1.

The mixing unit is configured to mix at least two gases to form a fresh gas mixture FG and is configured to provide this as a breathing gas mixture.

The breathing gas supply system has means—for example the gas conveying unit and/or in particular dosing valves or controllable proportional valves—for a controlled supply and/or dosing of an inspiratory breathing gas quantity and a device for controlling an expiratory breathing gas quantity—for example and in particular an expiratory valve (PEEP valve)—for controlling an expiratory breathing gas quantity.

The breathing gas supply system also has an oxygen dosing unit for controlled additional dosing of quantities of oxygen into the circuit system. This oxygen dosing unit can be integrated into the breathing gas supply system in such a way that the dosing of additional quantities of oxygen takes place at an inlet (gas inlet) of the gas conveying unit. Alternatively, the oxygen dosing unit can be integrated into the breathing gas supply system so that the dosing of additional quantities of oxygen takes place at the outlet (gas outlet) of the gas conveying unit.

The circuit system pneumatically connects the components of the breathing gas supply system, sensor components and other components with each other and provides an inspiratory connection and an expiratory connection to form pneumatic connections with the line system (tube system/pipe system).

In conventional embodiments, the anesthesia system can have further components for carrying out inhalation anesthesia and/or intravenously administered anesthesia, for example:
an anesthetic dosing unit,
an anesthetic gas delivery system,
a flush valve (scavenging valve/purge valve) arrangement,
an APL valve arrangement,
a breathing bag,
further sensors for pressure and flow rate measurement or for gas measurement.

The breathing bag is a reservoir in the circuit system that absorbs the quantities of breathing gas mixture exhaled by the patient.

The flush valve arrangement can be configured as a controllable dosing valve and is configured to flush parts or components of the circuit system or the circuit system.

The APL valve arrangement provides an adjustable pressure limitation valve (APL valve) in the circuit system, APL stands for “adjustable pressure limitation”. In a simplified configuration variant of the circuit system, the breathing bag, flush valve arrangement and/or APL valve arrangement can be omitted.

The anesthesia system is operated by a line system with the following components:
a patient connecting element (Y-piece),
an inspiratory breathing tube,
an expiratory breathing tube,
supplemental components

The line system is configured as a breathing tube system with the patient connecting element (Y-piece) for the supply of breathing gas quantities (inhalation gas) to the living being and for the continuation of breathing gas quantities (exhalation gas) away from the living being and thus serves the pneumatic and fluidic connection of the patient to the circuit system of the anesthesia system.

An inspiratory non-return valve is arranged in the inspiratory path of the line system and an expiratory non-return valve in the expiratory path in order to clearly define the direction of flow of inhaled and exhaled gas volumes in the circuit system and in the line system. The breathing tubes are connected to the circuit system on the device side with inspiratory and expiratory connections and connected to the patient connection element on the patient side.

An element for supplying gas to the patient, such as an endotracheal tube, a face mask or a tracheostoma (tracheal access), is connected to the patient connecting element.

The at least one flow sensor V1 of the sensor system is arranged on the breathing gas supply system, on the circuit system or on the line system in such a way that it continuously acquires (records/captures) at least one flow rate which can indicate quantities of breathing gas mixture supplied to the living being or quantities of breathing gas mixture carried away by the patient, or can indicate quantities of breathing gas mixture supplied or carried away over time and provide the control unit with measured values.

The at least one gas sensor G1 of the sensor system is arranged on the breathing gas supply system, on the circuit system or on the line system in such a way as to continuously detect at least one gas concentration which indicates a current concentration of oxygen supplied to the living being or to indicate a time course of current quantities of oxygen supplied and to provide the control unit with these as measured values.

The at least one pressure sensor P1 of the sensor system is arranged on the breathing gas supply system, on the circuit system or on the line system in such a way that the pressure sensor continuously records measured values which indicate at least one pressure level and provides these measured values to the control unit. The measured values indicate an airway pressure or a time course of an airway pressure.

In possible variants or embodiments of anesthesia systems or anesthesia devices, an anesthetic gas scavenging device may optionally be present or connected to the circuit system. Such an anesthetic gas scavenging device is used to quickly release a current gas mixture or used gas quantities from the circuit system, for example in situations where the user initiates an increase in the fresh gas quantity (FG) or when the O₂ flush function is activated, in order to achieve a rapid change in concentration ratios in the breathing gas mixture.

In possible variants or configurations of anesthesia systems or anesthesia devices, a breathing bag can be arranged on the circuit system or on the breathing gas supply system. Such a breathing bag can be used for manual ventilation or hand ventilation by the user—for example during certain phases of a surgical procedure. A valve (APL valve) can be used to limit the airway pressure supplied to the patient.

Possible suitable positions for arranging the at least one gas sensor G1 inside (operatively connected to) the breathing gas supply system, on the circuit system or on the line system are, for example:
on/in the circuit system in both the inspiratory path and the expiratory path,
on/in the line system both in the inspiratory path (insp. breathing tube) and in the expiratory path (exsp. breathing tube) as well as on the patient connection element (Y-piece),
on/in the breathing bag,
on/in the gas conveying unit or on components of the gas conveying unit, in particular on the housing of a piston drive, if the gas conveying unit is configured as a piston drive.

Possible suitable positions for arranging the at least one flow rate sensor V1 on (operatively connected to) the breathing gas supply system, on the circuit system or on the line system are, for example:
on/in the circuit system in both the inspiratory path and the expiratory path,
on/in the line system both in the inspiratory path (insp. breathing tube) and in the expiratory path (exsp. breathing tube) as well as on the patient connection element (Y-piece).

Possible suitable positions for arranging the at least one pressure sensor P1 on (operatively connected to) the breathing gas supply system, on the circuit system or on the line system are, for example:
on/in the circuit system in both the inspiratory path and the expiratory path,
in or on the breathing bag (BB),
on/in the gas conveying unit,
on/in the line system both in the inspiratory path (insp. breathing tube) and in the expiratory path (exsp. breathing tube) as well as on the patient connection element (Y-piece).

Representations of possible suitable positions for the arrangement of the at least one pressure sensor P1 as well as of further possible suitable positions for arrangements of pressure sensors are also shown on the basis of FIGS. 1, 2a and 2b. Representations of possible suitable positions for the arrangement of the at least one gas sensor G1 configured as an oxygen sensor as well as of further possible suitable positions for arrangements of gas sensors are also shown on the basis of FIGS. 1, 2a, 2b and 2c. Representations of possible suitable positions for the arrangement of the at least one flow rate sensor V1 as well as of further possible suitable positions for arrangements of flow rate sensors are also shown on the basis of FIGS. 1, 2a and 2b.

Possible suitable positions for positioning the breathing bag (BB) are on the breathing gas supply system or on the expiratory path of the circuit system.

Advantageously, the breathing bag (BB) can be arranged downstream of the expiratory non-return valve in the direction of flow.

A possible suitable position for connecting an anesthetic gas scavenging device (NGF, AGS—Anesthetic Gas Scavenging) is available, for example, on the expiratory path on the circuit system. With the mixing unit, the circuit system enables gases to be mixed to form a gas mixture that is suitable and intended for anesthesia and can be provided to a patient by the circuit system. In addition to air and oxygen, the gas mixture as so-called “fresh gas” also optionally consists of nitrous oxide and usually a volatile anesthetic (halothane, desflurane, enflurane, sevoflurane, isoflurane), which is introduced into the gas mixture by the anesthetic dosing unit (anesthetic vaporizer), for example in the form of a so-called vapor.

The gas conveying unit can be configured as a blower drive with a radial blower or as a piston drive with a piston and is configured to deliver quantities of the breathing gas mixture. The gas conveying unit is configured and intended to deliver the gas mixture to the patient.

Quantities of gas mixture are delivered to the patient in the circuit system via the inspiratory path, in which an inspiratory non-return valve is located, which prevents gases from flowing back from the patient into the inspiratory path.

The return flow from the patient takes place via the expiratory path into the circuit system.

An expiratory non-return valve is located in the expiratory path, which prevents gases from flowing back to the patient

Gas is supplied to the patient by means of the patient connection element, at which the inspiratory path is brought together and connected to an inspiratory breathing tube and the expiratory path to an expiratory breathing tube. During automatic ventilation, the gas conveying unit delivers a breathing gas mixture from the mixing unit through the circuit system into the inspiratory path as inspiratory gas to the patient during the inspiratory phase. During ventilation, the expiratory gas flows from the patient through the expiratory non-return valve during the expiratory phase back into the circuit system.

The control unit is configured and intended to organize, monitor, control or regulate the operation and/or sequence of the anesthesia system.

The control unit is preferably made up of components (μC, μP, PC) with associated operating system (OS), data memory (RAM, ROM, EEPROM) and SW code, software for sequence control, monitoring, control and regulation.

In at least some embodiments, further electronic elements such as components for signal acquisition (ADμC), signal amplification, analog and/or digital signal processing (ASIC), components for analog and/or digital signal filtering (DSP, FPGA, GAL, μC, μP), signal conversion (A/D converter) are assigned to the control unit or connected to the control unit. The control unit is configured to carry out a control and coordination of inspiratory and expiratory quantities of breathing gas mixture for ventilation of the living being on the basis of the measured values provided by the sensor system with the breathing gas supply system, in particular with the means for controlled dosing and the device (PEEP valve) for controlling the expiratory breathing gas quantity. An anesthetic dosing unit can be used to meter anesthetic gases into the inspiratory and expiratory quantities of breathing gas mixture and can be controlled and coordinated by the control unit, thus enabling anesthesia or inhalation anesthesia to be performed on the living being. Measured values of the at least one pressure sensor P1 and or measured values of the at least one flow rate sensor for controlling the timing of inspiration and expiration can be taken into account by the control unit. Based on the measured values of the at least one pressure sensor P1 and/or the at least one flow rate sensor, the control unit can determine the breathing phases with the sequence of inspiration phases and expiration phases even when the patient is breathing spontaneously. Taking into account the measured values of the at least one flow sensor V1 and the measured values of the at least one pressure sensor P1, the control unit can thus determine the quantities of breathing gas mixture supplied to the patient and the pressure levels thus given in inspiration (Pᵢₙₛₚ) and expiration (PEEP) and the sequence of inspiration and expiration, control, i.e. adjust, control or regulate the form of ventilation by controlling the gas conveying unit, for example by varying the speed of the radial blower of the blower drive or by changing the path of the piston of the piston drive accordingly i.e. adjust, control or regulate. During operation of the anesthesia system, the control unit continuously records measured values from the at least one pressure sensor P1 and the at least one flow sensor V1 with subsequent evaluation. The control unit is configured to determine the volumes of breathing gas mixture currently supplied to the living being, such as tidal volume VT or minute volume MV, based on the measured values of the at least one flow sensor V1. The control unit is also configured to determine the concentration of oxygen in the breathing gas mixture currently supplied to the living being on the basis of the measured values of the at least one gas sensor G1. The control unit is also configured to determine whether a special operating situation is present based on the measured values provided by the sensor system. In particular, measured values of the at least one flow rate sensor provided by the sensor system and measured values of the at least one gas sensor provided by the sensor system are used by the control unit and subjected to a comparison with lower threshold values. A special operating situation is characterized by a state in which the current oxygen concentration in the breathing gas quantities supplied to the living being is below a predetermined lower oxygen concentration threshold value. A special operating situation also exists if there is a state in which the current volume supplied to the living being is below a predetermined lower volume threshold value.

The control unit is configured to determine an individual ventilation situation on the basis of information provided on a tidal volume and/or a minute volume of the patient or on the basis of measured values provided by the sensor system on pressure ratios and/or flow rates of the breathing gas mixture. The control unit is configured to determine the oxygen concentration threshold value and/or the volume threshold value on the basis of the individual ventilation situation and to use the oxygen concentration threshold value and/or the volume threshold value to determine the special operating situation. In accordance with the invention, the control unit is configured to initiate an increase in a dosing quantity of oxygen into the circuit system if a special operating situation exists. By determining the special operating situation and the individual ventilation situation, a specific condition is identified during the course of anaesthetization of a patient and, adapted to the specific condition, an increase in a dosing quantity of oxygen is subsequently carried out by the control unit.

The advantage of this is that the dosing quantity of oxygen can be continuously adjusted taking into account the individual minute volume MV or the individual tidal volume VT of the patient when the special operating situation arises during the course of a surgical procedure under anesthesia. By including individual tidal volumes and/or minute volumes, it is thus possible to indirectly make advantageous and specific adjustments in concentration, quantity and/or duration when increasing the dosing quantity of oxygen with regard to patient categories such as adults, adolescents, children, infants as well as newborns or premature babies

In a preferred embodiment, the control unit can be configured to include a delay time duration—in particular an individual delay time duration—when the special operating situation occurs when the dosing quantity of oxygen in the circuit system is increased. In a preferred embodiment, the control unit can be configured to determine the delay time duration on the basis of the special operating situation or a user input. A delay period that can be configured by means of a user input offers the advantage that the user can adapt the delay period to the situation of the operation, taking into account his assessment and diagnostics based on further information, such as the patient's laboratory status or measured values for blood pressure, heart rate, etc. In addition, the control unit can be configured to determine the individual delay time based on the individual ventilation situation and/or the special operating situation. This offers the advantage that only short-term time intervals in which the special operating situation is present do not immediately initiate the increase of a dosing quantity of oxygen into the circuit system, but wait for the duration of the individual delay time, so that excessively frequent dosing of oxygen into the breathing system can be avoided. In addition, during the individual delay time, the user has the opportunity to notice the individual ventilation situation and/or the special operating situation and, if necessary, to make suitable settings on the anesthesia system himself/herself, i.e. without the initiative of the control unit, in order to enable a safe and comfortable operating state with a sufficient supply of oxygen for the living being. Advantageously, the individual delay time can also be selected depending on patient categories such as adults, adolescents, children, infants as well as newborns or premature babies.

In a further preferred embodiment, the control unit can be configured to include an indicator of a deviation of a current oxygen concentration from a lower oxygen alarm threshold (limit) in the determination of the individual delay time. The inclusion of the indicator of a deviation of a current oxygen concentration from a lower oxygen alarm threshold in the determination of the individual delay time also offers the advantage that, for example, adults can be allowed to tolerate a greater short-term shortfall than children and this can then be taken into account as an aspect in the determination of the individual delay time. In an advantageous way, the individual delay time can also be selected to suit the particular ventilation situation.

In a further preferred embodiment, the control unit can be configured to increase the quantity of oxygen dosed
after a predetermined delay time,
depending on a current error or alarm situation,
depending on an error situation in the sensor system,
depending on signals or data provided by the sensor system,
depending on a lower oxygen alarm threshold,
depending on information or data provided via a data interface.

These preferred embodiments offer the advantages of incorporating current error or operating situations of the anesthesia system or sensor system, the current alarm situation as well as external data for increasing the dosing quantity of oxygen.

In a further preferred embodiment, the control unit can be configured to initiate a dosing of oxygen into a fresh gas mixture FG at the mixing unit in order to increase the metered quantity of oxygen.

In a further preferred embodiment, the control unit can be configured to initiate a dosing of oxygen into the breathing gas supply system or the circuit system into a gas inlet or at a gas outlet on the gas conveying unit, on the absorber unit or on an anesthetic dosing unit by means of an oxygen dosing unit.

In a further preferred embodiment, the control unit can be configured to adapt a composition of the fresh gas mixture FG by means of the mixing unit in such a way that an increase in the proportion of oxygen in the fresh gas mixture FG results.

This preferred embodiment shows possibilities for increasing the proportion of oxygen in the fresh gas mixture FG for different application situations with different gas mixtures and the resulting operating states of anesthesia systems. Thus, when administering anesthesia with a mixture of nitrous oxide and oxygen as the fresh gas mixture FG, an increase in the concentration and quantity of oxygen in the fresh gas mixture FG can be achieved by switching—at least for a short time interval—to a mixture of air and oxygen as the fresh gas mixture FG or switching over by the control unit.

In a further preferred embodiment, the control unit can be configured to keep the total quantity of fresh gas mixture FG constant by increasing the dosing quantity of oxygen.

In a further preferred embodiment, the control unit can be configured to increase the total quantity of fresh gas mixture FG at the same time as increasing the dosing quantity of oxygen.

In a further preferred embodiment, the control unit can be configured to limit the total quantity of fresh gas mixture FG to a predetermined maximum level.

These preferred embodiments offer the advantage that both restrictions specified by the user-such as keeping the total quantity of fresh gas constant—or concessions—such as enabling an increase in the total quantity of fresh gas—can be taken into account by the control unit when increasing the dosing quantity of oxygen.

These preferred embodiments show possibilities for different configurations of anesthesia systems with breathing gas supply system, circuit system, mixing unit, gas conveying unit, absorber unit, anesthetic dosing unit, oxygen dosing unit and/or other components, each with suitable positions for the dosing of oxygen.

In a further preferred embodiment, the control unit can be configured to include information provided for determining the individual ventilation situation—in particular information provided at the data interface and/or at an input interface—in relation to a weight, a gender, an age or a body size of the living being as well as information on minute volume MV and/or information on tidal volume VT or a patient category of the living being. These preferred embodiments offer the advantage that information available from a data network or network (LAN, WLAN, Ethernet, serial interfaces) can be included in the determination of the individual ventilation situation. In this way, information on measurements or examination results, diagnostic or therapeutic information or the current medication can also be included in the determination of the individual ventilation situation.

In a further preferred embodiment, the control unit can be configured to increase the dosing quantity of oxygen depending on the individual ventilation situation with at least two stages of oxygen concentration levels.

In a further preferred embodiment, the control unit can be configured to limit the increase in the dosing quantity of oxygen to a predetermined maximum concentration value of oxygen in the breathing circuit supply system. An at least two-stage or even multi-stage increase, as well as an increase in the dosing quantity of oxygen limited to a maximum concentration value of oxygen depending on the individual ventilation situation, can enable a gradual approximation of the increase.

This can also prevent, for example, an increase in the oxygen dosing quantity with too large an excess of fresh gas mixture and thus a waste of gas and/or anesthesia.

In a further preferred embodiment, the control unit can be configured to provide an output signal or an alarm signal to an output unit or to a data interface, which indicates the individual ventilation situation, the special operating situation, the current oxygen concentration, the individual delay time duration, the dosing quantity of oxygen or the quantity of fresh gas mixture FG. This preferred embodiment offers several advantages for informing the user of the function of the anesthesia system and the operating mode of the anesthesia system.

In a further preferred embodiment, at least components of the sensor system—for example as pressure sensors—can be arranged on the line system, preferably on the patient connecting element (Y-piece), to detect a pressure in the airways of the living being. In a further preferred embodiment, at least components of the sensor system—for example as a gas sensor in the form of an infrared-optical carbon dioxide sensor—can be arranged close to the patient on the line system, preferably on the patient connecting element (Y-piece), in order to detect the quantities of carbon dioxide exhaled by the living being.

In a further preferred embodiment, at least components of the sensor system-such as a gas sensor in the form of an electrochemical or paramagnetic oxygen sensor—can be arranged close to the patient on the line system, preferably on the patient connection element (Y-piece), to detect the quantities of oxygen inhaled by the living being.

In a further preferred embodiment, components of the sensor system—for example as a flow rate sensor—can be arranged on the line system, preferably on the patient connecting element (Y-piece), to record a balance of the quantities of gas mixture inhaled and/or exhaled again by the living being. In a further preferred embodiment, the anesthetic dosing unit can be arranged downstream of the mixing unit, in particular at an outlet of the mixing unit, in or on the anesthesia system or can be associated with the anesthesia system. The anesthetic dosing unit is configured to meter an anesthetic into the fresh gas or into the breathing gas mixture. In a further preferred embodiment, an anesthetic gas scavenging unit can be arranged in or on the anesthesia system downstream of the device for the expiratory breathing gas volume (expiratory valve, PEEP valve) or can be associated with the anesthesia system. The anesthesia gas scavenging unit is configured to scavenge gas volumes out of the anesthesia system to the outside.

These embodiments show how anesthesia systems can be configured in a variety of ways in order to put the functions and advantages for patient and user according to the invention into practice.

In a further preferred embodiment, the control unit can be configured to terminate a state with an increased dosing quantity of oxygen or the special operating situation after a predetermined maximum activation period as a function of an operating action by a user, as a function of signals or data provided by the sensor system or as a function of information or data provided by means of a data interface. These embodiments show ways in which anesthesia systems can be configured in a variety of ways to return to an operating state without increased oxygen dosing after an increase in the dosing quantity of oxygen.

The present invention will now be explained in more detail with the aid of the following figures and the associated figure descriptions, without limiting the general concepts of the invention. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a basic structure of an anesthesia device according to the invention;

FIG. 2a is a schematic view showing a variant of the anesthesia device with piston drive;

FIG. 2b is a schematic view showing a variant of the anesthesia device with blower drive (radial fan, blower);

FIG. 2c is a schematic view showing an alternative variant as shown in FIG. 2a with a piston drive;

FIG. 3 is a flow diagram showing a procedure for determining a special operating situation.

### DESCRIPTION OF PREFERRED EMBODIMENTS

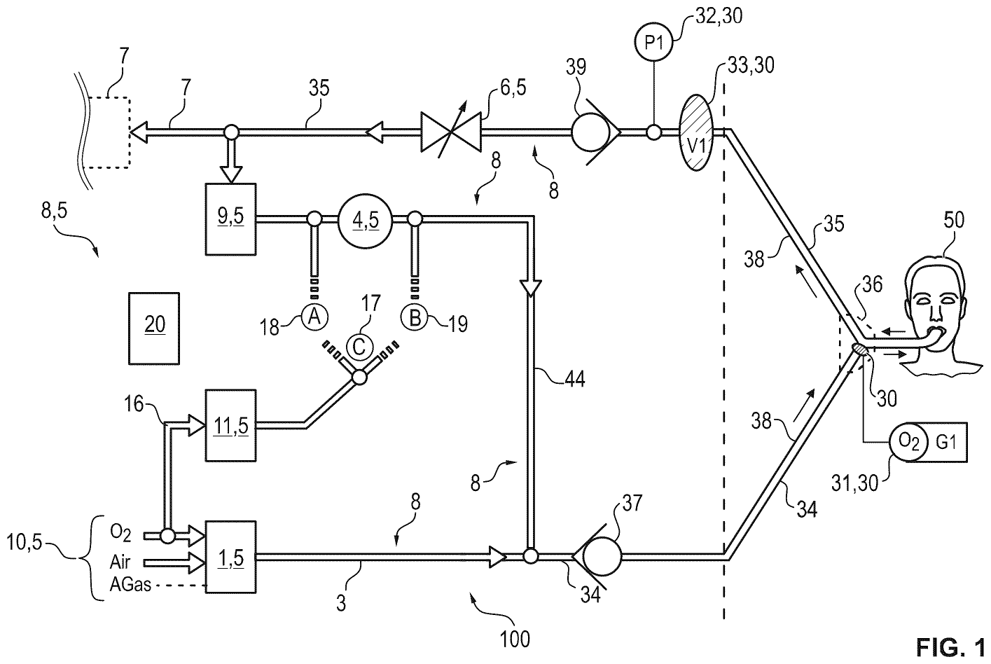

Referring to the drawings, Identical elements in FIGS. 1, 2a, 2b, 2c, 3 are designated with identical reference numerals in FIGS. 1, 2a, 2b, 2c, 3.

FIG. 1 shows the basic structure of an anesthesia device 100 with a breathing gas supply system 5, a mixing unit 1, a gas conveying unit 4, an expiration valve 6, a circuit system 8 and an absorber unit 9. Gases such as oxygen, air or nitrous oxide are supplied to the mixing unit 1 via a gas supply 10. The absorber unit 9 makes it possible to remove quantities of carbon dioxide from the circulation system 8.

The circuit system 8 has an inspiratory path 34 and an expiratory path 35 as well as an inspiratory non-return valve 37 and an expiratory non-return valve 39. The inspiratory and expiratory paths 34 and 35 are routed to a patient 50 via a connecting element (Y-piece) 36 via a line system 38, thus realizing the patient's pneumatic connection to the anesthesia device 100.

An additional oxygen dosing unit 11 with an input 16 is connected or coupled to the gas supply 10, the output C 17 of which can be connected to the gas conveying unit 4 at two different positions A 18 or B 19. The difference between the two positions A 18, B 19 is that when C 17 is connected to position A 18, additional quantities of oxygen are fed into the inlet of the gas conveying unit 4, whereas when C 17 is connected to position B 19, additional quantities of oxygen are fed into the circuit system 8 at the outlet of the gas conveying unit 4. A fresh gas line 3 feeds quantities of fresh gas (FG) from the mixing unit 1 into the inspiratory path 34 of the breathing system 8. A rebreathing line (ventilation line) 44 connects the quantities of gas freed from carbon dioxide by means of the absorber unit 9 into the inspiratory path 34.

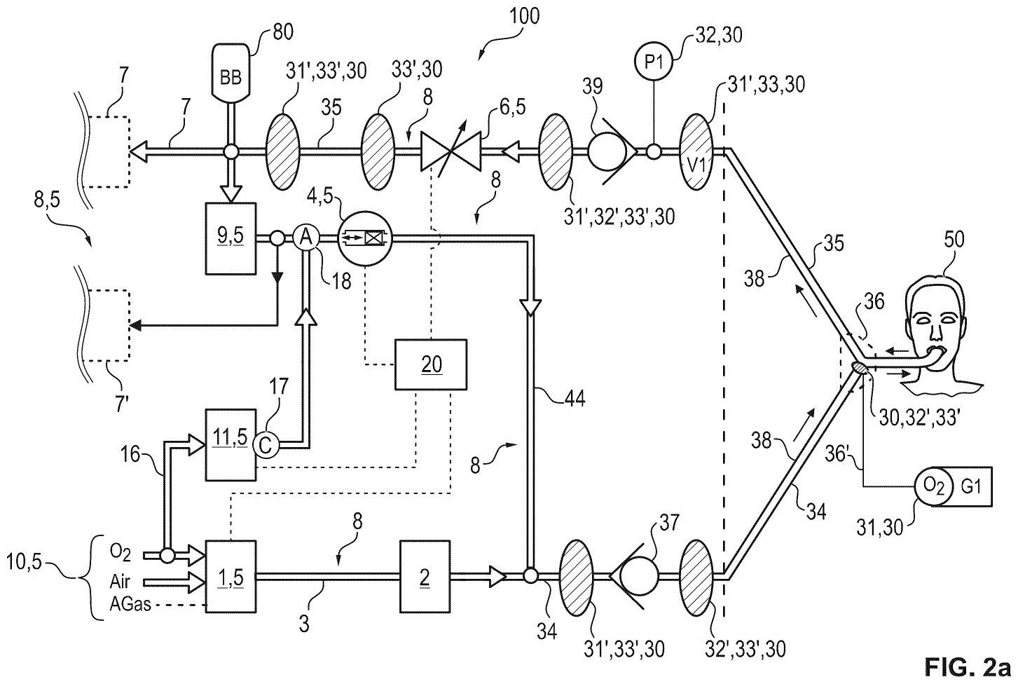

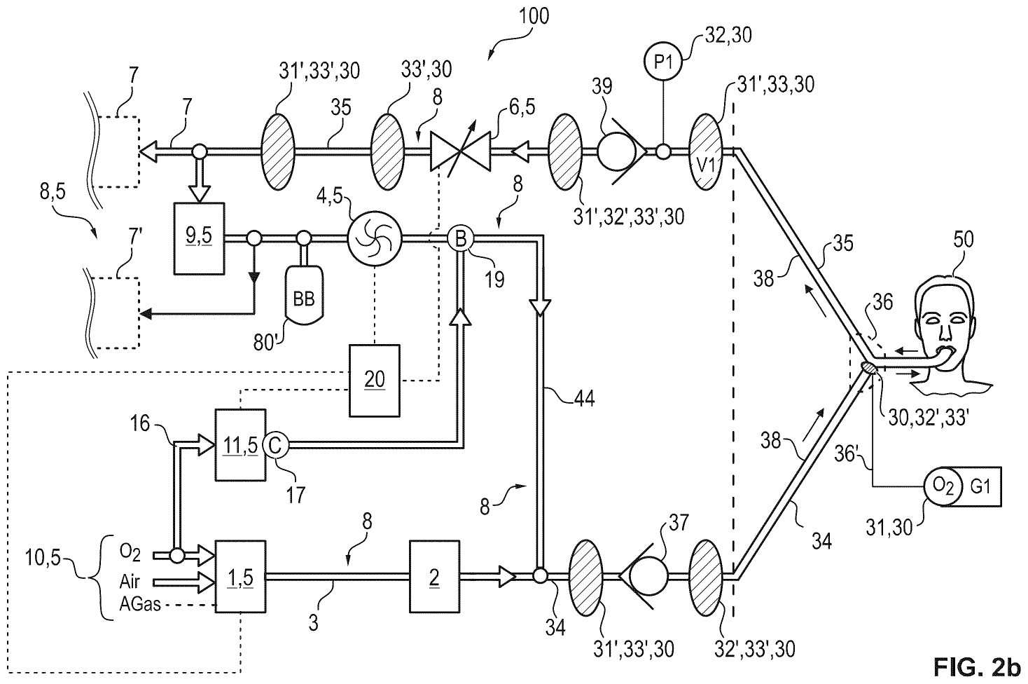

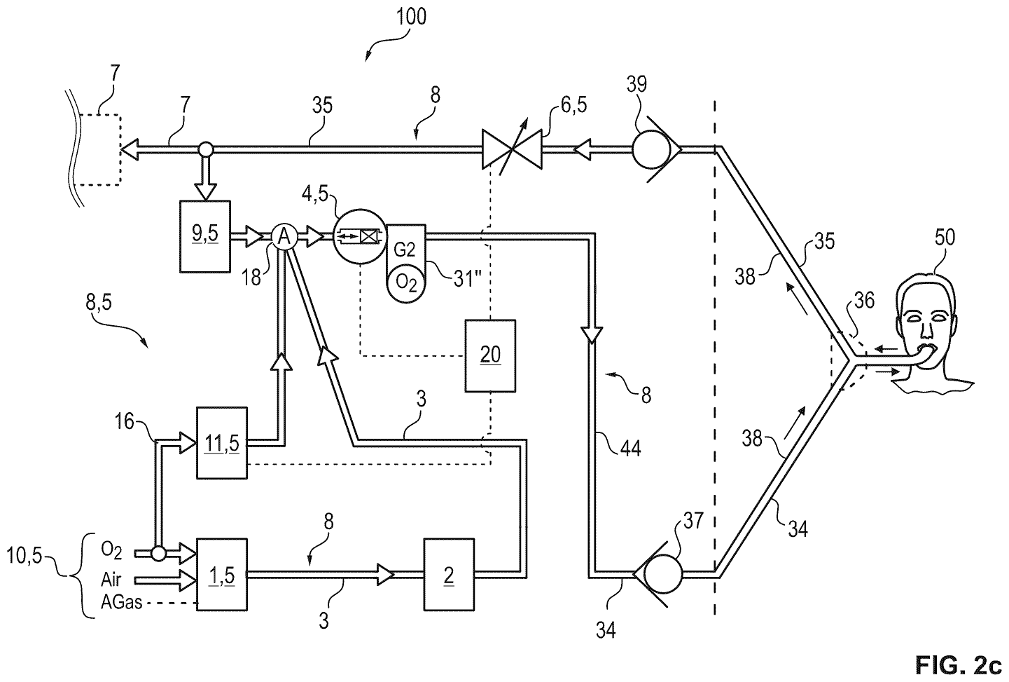

A connection of C 17 to position A 18 (inlet of the gas conveying unit 4) is a preferred embodiment of the anesthesia device 100 in such a way that the gas conveying unit 4 is configured as a piston drive (piston) (FIG. 2a). A connection from C 17 to position B 19 (outlet of the gas conveying unit 4) is a preferred configuration of the anesthesia device 100 such that the gas conveying unit 4 is configured as a blower drive (FIG. 2b).

The anesthesia device 100 has a sensor system 30 with at least one pressure sensor P1 32, at least one flow rate sensor V1 33 and at least one gas sensor G1 31 in the form of an oxygen sensor. Further sensors 30 with alternative or additional pressure sensors 32′, flow rate sensors 33′ or gas sensors 31′ can supplement the anesthesia device 100, as will be explained in more detail in FIGS. 2a, 2b, 2c. A control unit 20 is used to control the anesthesia device 100, as will be explained in more detail in FIGS. 2a, 2b, 2c. The control by the control unit 20 can be configured with aspects of coordination, sequence control, status control and/or regulation.

FIG. 2a shows the basic structure of an anesthesia device 100 as shown in FIG. 1, wherein the gas conveying unit 4 is configured as a piston drive. FIG. 2b shows the basic structure of an anesthesia device 100 according to FIG. 1, wherein the gas conveying unit 4 is configured as a blower drive. FIG. 2c shows a variant of a basic structure of an anesthesia device 100 as shown in FIG. 2a. FIGS. 2a and 2b are explained in more detail below in a joint description of the figures. Identical elements in FIGS. 2a and 2b are designated with identical reference numbers in both FIGS. 2a and 2b.

FIGS. 2a and 2b show an anesthetic gas dosing unit 2 arranged in the inspiratory path 34 of the circuit system 8. The anesthetic gas dosing unit 2 can be attached to the anesthesia device 100 as an additional component or can be configured as an integral element of the anesthesia device 100. A fresh gas line 3 feeds quantities of fresh gas (FG) from the mixing unit 1 via an anesthetic gas dosing unit 2 into the inspiratory path 34 of the breathing system 8. A rebreathing line 44 connects the supply of gas quantities, freed from carbon dioxide by means of the absorber unit 9, into the inspiratory path 34. In FIG. 2a, the inlet 18 of the gas conveying unit 4 (piston drive) is selected as the feed point A for the oxygen additive dosing 11. In FIG. 2b, output 19 of gas conveying unit 4 (blower) is selected as feed point B for the oxygen additive dosing 11.

Nevertheless, it should also be mentioned that in practical implementations, both feed points 18, 19 can be selected for a gas conveying unit 4 in the form of a blower drive as well as a piston drive. A control unit 20 is arranged in the anesthesia device 100, which is configured and intended to control the piston drive or the blower drive of the gas conveying unit 4, the expiration valve 6 and the oxygen additive dosing 11 for performing anesthesia on a patient 50. In addition, the control unit 20—as shown in FIGS. 2a, 2b, 2c—can be configured and intended to control the mixing unit 1 in order to adjust a gas composition of a fresh gas mixture (FG). In FIGS. 2a and 2b, at least one gas sensor G1 31 is arranged in the circuit system 8 or on the line system 38. In these two figures, FIGS. 2a and 2b, the at least one gas sensor G1 31 is configured as an O₂ gas sensor 31 close to the patient with an aspirating measuring functionality by means of a measuring gas line 36′ for a measurement at the patient connecting element (Y-piece) 36. In optional embodiments, the at least one gas sensor 31 can also be arranged downstream of the inspiratory non-return valve 37 in the direction of flow. In FIGS. 2a and 2b, at least one pressure sensor P1 32 is arranged in or on the circuit system 8.

In FIGS. 2a and 2b, at least one flow rate sensor V1 33 is arranged in or on the circuit system 8. FIG. 2a shows a breathing bag BB 80 arranged in or on the circuit system 8. In FIG. 2b, the breathing bag BB 80′ is shown in an alternative position. Alternative and/or optional arrangements of sensors 30 with the respective sensors 31′, 32′, 33′ are shown at positions in the anesthesia device 100 that are particularly indicated by hatched ellipses. Alternative and/or optional arrangements of gas sensors are shown at positions as indicated by the reference number 31′.

Such an optional gas sensor system 31′ can additionally and/or optionally supplement the sensor system 30 (FIG. 1). Alternative and/or optional arrangements of pressure sensors are given at positions as indicated in each case by the reference numeral 32′. Such an optional pressure sensor system 32′ can additionally and/or optionally supplement the sensor system 30 (FIG. 1). Alternative and/or optional arrangements of flow rate sensors are given at positions as indicated by the reference numeral 33′. Such an optional flow rate sensor system 33′ can additionally and/or optionally supplement the sensor system 30 (FIG. 1). FIGS. 2a and 2b show an anesthetic gas scavenging unit 7 arranged on the circuit system 8. An alternative arrangement of an anesthetic gas scavenging unit is shown—as additionally illustrated in FIGS. 2a and 2b—at a position designated by the reference number 7′.

FIG. 2c shows an embodiment variant according to FIG. 2a with a gas conveying unit 4 configured as a piston drive. Identical elements in FIGS. 2a, 2b and 2c are designated with identical reference numbers in both FIGS. 2a, 2b and 2c. The sensor system 30 and also the optional sensor systems 31′, 32′, 33′ are not shown by means of hatched symbols in this FIG. 2c for the sake of clarity; suitable positions of the optional sensor systems 31′, 32′, 33′ in FIG. 2c are possible and can be realized accordingly, as shown and described in FIGS. 1 and 2a. In this FIG. 2c, the gas sensor system 30 (FIG. 1) is configured as an oxygen gas sensor G2 31, which is arranged on the piston drive (piston) of the gas conveying unit 4 or on the housing of the gas conveying unit 4.

In this FIG. 2c—in contrast to FIGS. 1, 2a and 2b—the fresh gas line 3 leads from the outlet of the anesthetic gas dosing unit 2 to the inlet of the gas conveying unit 4.

A rebreathing line (return line) 44 enables the supply of gas quantities freed from carbon dioxide by the absorber unit 9 and quantities of fresh gas (FG) into the inspiratory path.

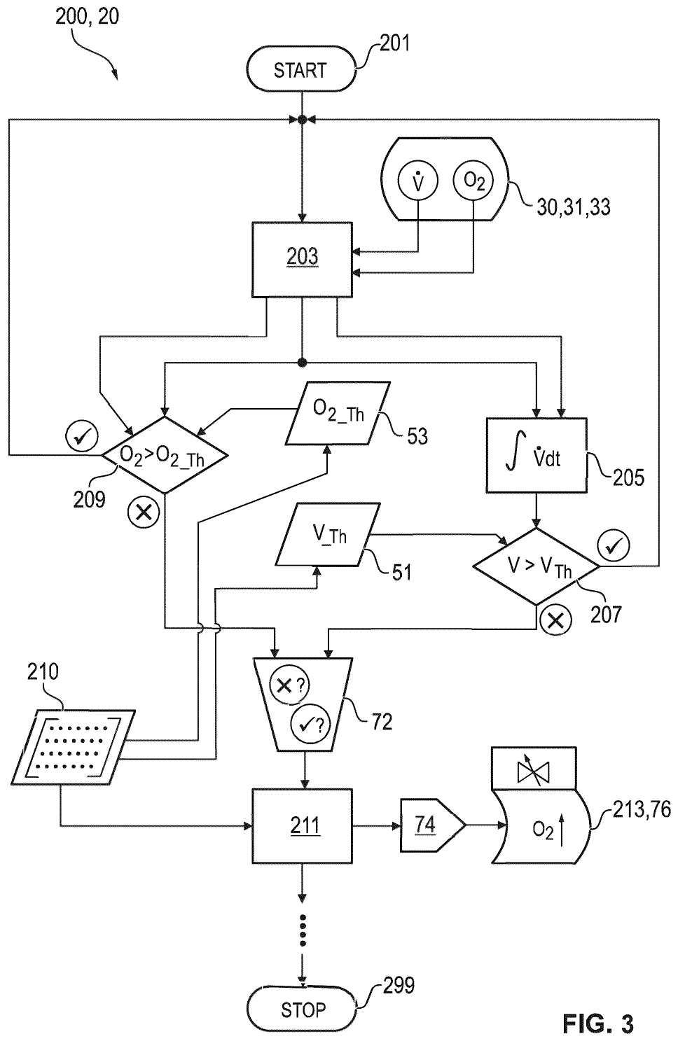

FIG. 3 shows a sequence 200 for activating the oxygen supplement dosage 11 (FIG. 1) by a control unit 20 in special situations during the performance of ventilation or anesthesia by an anesthesia device 100 (FIG. 1). Beginning with a start 201, a continuous measured value acquisition 203 of measured values of a sensor system 30, which comprises at least one gas sensor G1 31 configured as an oxygen sensor and a flow rate sensor V1 33, takes place.

The measured value acquisition of pressure measured values, which are necessary for performing ventilation and/or anesthesia operation, is not shown in order to maintain clarity in the sequence 200. Following the measured value acquisition 203, signal processing 205 of the flow rate measured values and integral formation for determining the tidal volume VT and/or minute volume MV and a threshold value comparison 207 with a volume threshold value 51 as well as a threshold value comparison 209 with a concentration threshold value 53 take place.

The volume threshold 51 can be adjusted based on information 210 provided on tidal volumes and/or minute volumes, for example with regard to patient categories such as age (adults, children, infants), gender, height, weight, previous illnesses and clinical picture. The results of the two comparisons 205, 207 are combined to determine 72 whether an individual ventilation situation exists. As soon as the individual ventilation situation 72 has been determined, the results of the two comparisons 207, 209 are consolidated 211 using the information 210 to determine whether a special operating situation 74 exists.

The special operating situation 74 exists if the comparisons 205, 207 have shown that the current tidal volume VT and/or the current minute volume MV are below the volume threshold value 51—now adjusted by means of the individual ventilation situation—and the current oxygen concentration is also below the concentration threshold value 53.

If the special operating situation 74 exists, an activation 213 of the O₂ additional dosing 11 (FIG. 1) takes place in order to effect an increase 76 of a quantity or concentration of oxygen in the breathing circuit 8 (FIG. 1). Subsequently, the sequence 200 comes to an end 299 to an activation of the additional oxygen dosage, and the performance of the anesthetic operation is continued.

While specific embodiments of the invention have been shown and described in detail to illustrate the application of the principles of the invention, it will be understood that the invention may be embodied otherwise without departing from such principles.

### LIST OF REFERENCE NUMBERS

1 Mixing unit
2 Anesthetic dosing unit (optional)
3 Fresh gas line
4 Gas conveying unit
5 Breathing gas supply system
6 Device for expiratory dosing, expiratory valve
7 Anesthesia gas scavenging unit, AGS, NFV, internal, external (optional)
7′ Optional positions for anesthetic gas scavenging unit on the breathing gas supply system or on the circuit system
8 Circuit system
9 Absorber unit, CO₂ absorber
10 Gas supply
11 Oxygen additive dosing, O₂ additive dosing (O₂)
16 Oxygen additive dosing input with connection to the gas supply
17 Output C of the oxygen additive dosing with connection to the gas conveying unit
18 Feed point A at the inlet of the gas conveying unit
19 Feed point B at the outlet of the gas conveying unit
20 Control unit
30 Sensors
31 Near-patient O₂ gas sensor G1, G2
31′ Optional positions for O₂ gas sensors
31″ O₂ gas sensor G2 on the piston actuator
32 Pressure sensor P1
32′ Optional positions for pressure sensors
33 Flow rate sensor V1
33′ Optional positions for flow rate sensors
34 Inspiratory path
35 Expiratory path
36 Connecting element, Y-piece,
36′ Sample gas line (optional)
37 Non-return valve, inspiratory
38 Line system, breathing line system
39 Non-return valve, expiratory
44 Rebreathing line
50 Living being, patient
51 Volume threshold
53 Concentration threshold
72 Determination of an individual ventilation situation
74 Special operating situation
76 Increasing a dosing quantity
80 Breathing bag BB
100 Anesthesia device, anesthesia system
200 Procedure (process)
201 Start
203 Measured value acquisition by the control unit
205 Signal processing of flow measurement values and integral calculation to determine tidal volume VT and/or minute volume MV
207 Threshold value comparison volume:
VT_actual < > VT_target or MV_actual < > MV_target
209 Threshold value comparison concentration:
   O₂_actual < > O₂_nominal
210 Information on tidal volume, minute volume, patient categories
211 Consolidation of the threshold value comparisons
213 Activation of additional O2 dosing (O₂)
299 End, stop

What is claimed is:

1. An anesthesia system to anesthetize a living being with an optimized oxygen supply, the anesthesia system comprising:
a breathing gas supply system, the breathing gas supply system comprising: a circuit system, the circuit system comprising an absorber unit configured to remove carbon dioxide from the circuit system and a gas conveying unit; a mixing unit, the mixing unit being configured to mix at least two gases provided by a gas supply to form a quantity of a fresh gas mixture and to feed the fresh gas mixture into the circuit system, wherein the breathing gas supply system is configured by means of the gas conveying unit to convey the fresh gas mixture and to provide a breathing gas mixture for the living being and control a provision and/or a dosing of an inspiratory breathing gas quantity into the circuit system; and a breathing gas quantity device configured to control an expiratory breathing gas quantity into the circuit system;
a line system operatively connected to the circuit system, which is configured as a breathing tube system with a connecting element, the breathing tube system being configured to supply quantities of breathing gas to the living being and to convey quantities of breathing gas away from the living being;
a sensor system, which comprises:
one or more gas sensors operatively connected to the circuit system or operatively connected to the line system such that at least one gas concentration is continuously acquired as oxygen supplied measured values, wherein the oxygen supplied measured values indicate a current concentration of oxygen supplied to the living being or a time course of current quantities of oxygen supplied;
one or more pressure sensors operatively connected to the circuit system or operatively connected to the line system such that at least one pressure level is continuously acquired as airway pressure measured values, wherein the airway pressure measured values indicate an airway pressure or a time course of an airway pressure; and
one or more flow rate sensors operatively connected to the circuit system or operatively connected to the line system such that at least one flow rate is continuously acquired as flow rate measured values, wherein the flow rate measured values indicate quantities of breathing gas mixture supplied to the living being or a time course of supplied quantities of breathing gas mixture; and
a control unit, which is configured:
to carry out a control and coordination of inspiratory and expiratory quantities of breathing gas mixture for ventilation of the living based on the measured values provided by the sensor system
to determine, based on the measured values provided by the sensor system, whether a special operating situation is present, which is characterized by a state in which a current oxygen concentration in breathing gas quantities supplied to the living being is below a predetermined lower oxygen concentration threshold value, and/or by a condition existing in which an actual volume supplied to the living being is below a predetermined lower volume threshold value;
to determine an individual ventilation situation based on information provided on a tidal volume and/or on a minute volume of the patient and/or based on the measured values provided by the sensor system for pressure ratios and/or flow rates of the breathing gas mixture;
to determine the oxygen concentration threshold value and/or the volume threshold value based on the individual ventilation situation and to apply oxygen concentration threshold value and/or the volume threshold value to determine the special operating situation;
to initiate an increase in a dosing quantity of oxygen into the circuit system as a function of the special operating situation.

2. An anesthesia system according to claim 1,
wherein the control unit is configured to include a delay time period, comprising an individual delay time period, upon the special operating situation being present and upon activation of the increase in the dosing quantity of oxygen in the circuit system,
wherein the control unit is configured to set the delay time duration based on the special operating situation or based on a user input and/or the control unit is configured to determine the individual delay time duration based on the individual ventilation situation and/or the special operating situation.

3. An anesthesia system according to claim 2, wherein the control unit is configured to include in the determination of the individual delay time an indicator of a deviation of a current oxygen concentration from a lower oxygen alarm threshold.

4. An anesthesia system according to claim 3, wherein the control unit is configured to initiate the increase in the dosing quantity of oxygen:
after the predetermined delay time;
depending on a current error or alarm situation;
depending on an error situation of the sensor system;
depending on signals or data provided by the sensor system;
depending on a lower oxygen alarm threshold;
depending on information or data provided via a data interface.

5. An anesthesia system according to claim 1, wherein the control unit is configured to carry out the increase in the dosing amount of oxygen such that:
the dosing of oxygen into a fresh gas mixture at the mixing unit is initiated; and/or
the dosing of oxygen into the breathing gas supply system is initiated by means of an oxygen dosing unit into a gas inlet or by means of a gas outlet on the gas conveying unit, or by means of the absorber unit or by means of an anesthetic dosing unit; and/or
the mixing unit is adjusted as to a composition of the fresh gas mixture such that a proportion of oxygen in the fresh gas mixture is increased.

6. An anesthesia system according to claim 1,
wherein the control unit is configured to keep a total quantity of fresh gas mixture constant with the increase in the dosing quantity of oxygen, or
wherein the control unit is configured to increase the total quantity of fresh gas mixture with the increase in the dosing quantity of oxygen, or
wherein the control unit is configured to limit the total quantity of fresh gas mixture to a predetermined maximum level with the increase in the dosing quantity of oxygen.

7. An anesthesia system according to claim 1, wherein the control unit is configured to include additional information to determine the individual ventilation situation, wherein the additional information relates to one or more of a weight of the living being, to a gender of the living being, to an age of the living being, to a body size of the living being, and a patient category of the living being, as well as the information based on the tidal volume and/or the minute volume.

8. An anesthesia system according to claim 7, further comprising a data interface and/or an input interface wherein the additional information is input at the data interface and/or at an input interface.

9. An anesthesia system according to claim 1, wherein the control unit is configured:
to increase the dosing quantity of oxygen depending on the individual ventilation situation with at least two stages of oxygen concentration levels; and/or
to limit the increase in the dosing quantity of oxygen to a predetermined maximum concentration value of oxygen in the circuit system.

10. An anesthesia system according to claim 1, further comprising an output unit and/or a data interface,
wherein the control unit is configured to provide an output signal and/or an alarm signal to the output unit or to the data interface,
wherein the output signal and/or the alarm signal indicates the individual ventilation situation, the special operating situation, the current oxygen concentration, the individual delay time duration, the dosing quantity of oxygen and/or the quantity of fresh gas mixture.

11. An anesthesia system according to claim 1, wherein the control unit is configured to terminate a state with an increased dosing amount of oxygen or the special operating situation at least one of:
after a predetermined maximum activation time period;
depending on an operating action by a user;
depending on signals or data provided by the sensor system; and
depending on information or data provided via a data interface.
